(19) United States
(12) Patent Application Publication
(10) Pub. No.: US 2012/0190891 A1
(43) Pub. Date: Jul. 26, 2012

(54) SYSTEMS AND METHODS FOR PROCESSING LIPID FEEDSTOCKS

(76) Inventors: Clayton V. McNeff, Andover, MN (US); Bingwen Yan, Shoreview, MN (US); Daniel Thomas Nowlan, Hugo, MN (US)

(21) Appl. No.: 13/301,316

(22) Filed: Nov. 21, 2011

Related U.S. Application Data
(60) Provisional application No. 61/415,704, filed on Nov. 19, 2010.

(51) Int. Cl. C07C 29/09; C07C 27/02

(52) U.S. Cl. 562/589; 568/858

(57) ABSTRACT

Embodiments of the invention include processing lipid feedstocks into various products. In an embodiment, the invention includes a method of processing a lipid feedstock including combining the lipid feedstock with water to form a reaction mixture; and contacting the reaction mixture with a catalyst, the catalyst including a metal oxide. Methods can be used to form polyols, diols, and carboxylic acids, amongst others. Other embodiments are also included herein.

This application claims the benefit of U.S. Provisional Application No. 61/415,704, filed Nov. 19, 2010, the content of which is herein incorporated by reference in its entirety.

### FIELD OF THE INVENTION

The present invention relates to processing lipid feedstocks. More specifically, the present invention relates to processing lipid feedstocks into various products.

### BACKGROUND OF THE INVENTION

Many chemical compounds have broad industrial applications. As one example, lactic acid (2-hydroxypropanoic acid), an alpha hydroxy acid, can serve as the monomer for formation of polylactic acid (PLA), a biodegradable polymer with many applications. Lactic acid is also used as an ingredient in various foods and beverages. In addition, lactic acid has various used in the detergents industry. As another example, propylene glycol, an example of a diol, has many uses including as a solvent in various pharmaceuticals, as a humectant, as an emulsifier, and many others. As yet another example, glycerin as an example of a polyol, has many uses in the food industry, in various pharmaceuticals, and products such as anti-freeze.

However, a need remains for environmentally friendly systems and methods for producing useful compounds such as these, amongst others.

### SUMMARY OF THE INVENTION

Embodiments of the invention include processing lipid feedstocks into various products. In an embodiment, the invention includes a method of processing a lipid feedstock including combining the lipid feedstock with water to form a reaction mixture; and contacting the reaction mixture with a catalyst, the catalyst including a metal oxide.

In an embodiment, the invention includes a method of producing a polyol including combining a lipid feedstock with water to form a reaction mixture, and contacting the reaction mixture with a catalyst, the catalyst including a metal oxide.

In an embodiment, the invention includes a method of producing glycerin including combining a lipid feedstock with water to form a reaction mixture; and contacting the reaction mixture with a catalyst, the catalyst including a metal oxide.

In an embodiment, the invention includes a method of producing a diol including combining a lipid feedstock with water to form a reaction mixture; and contacting the reaction mixture with a catalyst, the catalyst including a metal oxide.

In an embodiment, the invention includes a method of producing propylene glycol including combining a lipid feedstock with water to form a reaction mixture; and contacting the reaction mixture with a catalyst, the catalyst including a metal oxide.

In an embodiment, the invention includes a method of producing a carboxylic acid including combining a lipid feedstock with water to form a reaction mixture; and contacting the reaction mixture with a catalyst, the catalyst including a metal oxide.

In an embodiment, the invention includes a method of producing an alpha hydroxy acid including combining a lipid feedstock with water to form a reaction mixture; and contacting the reaction mixture with a catalyst, the catalyst including a metal oxide.

In an embodiment, the invention includes a method of producing lactic acid including combining a lipid feedstock with water to form a reaction mixture; and contacting the reaction mixture with a catalyst, the catalyst including a metal oxide.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE FIGURES

The invention may be more completely understood in connection with the following drawings, in which:

FIG. 1 is a schematic view of reactor system according to an embodiment.

FIG. 2 is a GC chromatogram of samples ST36-58AB, ST36-60BC and ST36-61DE.

FIG. 3 is a GC chromatogram of samples ST36-62CD, ST36-63CD and ST36-63DE.

FIG. 4 is a NMR analysis graph of glycerin in sample ST36-8AB.

FIG. 5 is a NMR analysis graph of glycerin in sample ST36-60BC.

FIG. 6 is a NMR analysis graph of glycerin in sample ST36-62CD.

FIG. 7 is a NMR analysis graph of glycerin in sample ST36-63DE.

FIG. 8 is a ¹H-NMR analysis graph of crude glycerol solution pre-distillation.

FIG. 9 is a ¹H-NMR analysis graph of distillation pot showing pure glycerin.

FIG. 10 is a ¹H-NMR analysis graph of distillate showing lactic acid and propylene glycol and glycerin resonances.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

As demonstrated herein, the synthesis of products such as polyols (glycerin), carboxylic acids (lactic acid), and diols (propylene glycol) from water and a lipid feedstock can be efficiently catalyzed by certain metal oxides. While not intending to be bound by theory, it is believed that the use of metal oxides to catalyze the synthesis of such products can offer various advantages. For example, metal oxide catalysts used with embodiments of the invention are extremely durable making them conducive to use in many different potential processing steps. In addition, such metal oxide catalysts can be reused many times, making this approach cost effective. In addition, processes herein can be highly efficient in contrast to other techniques such as fermentation based techniques used for the production of materials such as lactic acid.

It will be appreciated that as used herein the terms “glycerol”, “glycerine” and “glycerin” are used herein interchangeably.

Metal oxide catalysts used with embodiments of the invention can include metal oxides with surfaces including Lewis acid sites, Bronsted base sites, and Bronsted acid sites. By definition, a Lewis acid is an electron pair acceptor. A Bronsted base is a proton acceptor and a Bronsted acid is a proton donor. Metal oxide catalysts of the invention can specifically include zirconia, alumina, titania and hafnia. Metal oxide catalysts of the invention can also include silica clad with a metal oxide selected from the group consisting of zirconia, alumina, titania, hathia, zinc oxide, copper oxide, magnesium oxide and iron oxide. In some embodiments, the metal oxide catalyst can be of a single metal oxide type. By way of example, in some embodiments, the metal oxide catalyst is substantially pure titania. In some embodiments, the metal oxide catalyst is substantially pure alumina. Metal oxide catalysts of the invention can also include mixtures of metal oxides, such as mixtures of metal oxides including zirconia, alumina, titania and/or hafnia. Of the various metal oxides that can be used with embodiments of the invention, zirconia, titania, alumina and hafnia are advantageous as they are very chemically and thermally stable and can withstand very high temperatures and pressures as well as extremes in pH. Titania and alumina are advantageous because of the additional reason that they are less expensive materials.

Metal oxides of the invention can include metal oxide particles clad with carbon. Carbon clad metal oxide particles can be made using various techniques such as the procedures described in U.S. Pat. Nos. 5,108,597; 5,254,262; 5,346,619; 5,271,833; and 5,182,016, the contents of which are herein incorporated by reference. Carbon cladding on metal oxide particles can render the surface of the particles more hydrophobic.

Metal oxides of the invention can also include polymer coated metal oxides. By way of example, metal oxides of the invention can include a metal oxide coated with polybutadiene (PBD). Polymer coated metal oxide particles can be made using various techniques such as the procedure described in Example 1 of U.S. Pub. Pat. App. No. 2005/0118409, the contents of which are herein incorporated by reference. Polymer coatings on metal oxide particles can render the surface of the particles more hydrophobic.

Metal oxide catalysts of the invention can be made in various ways. As one example, a colloidal dispersion of zirconium dioxide can be spray dried to produce aggregated zirconium dioxide particles. Colloidal dispersions of zirconium dioxide are commercially available from Nyacol Nano Technologies, Inc., Ashland, Mass. The average diameter of particles produced using a spray drying technique can be varied by changing the spray drying conditions. Examples of spray drying techniques are described in U.S. Pat. No. 4,138,336 and U.S. Pat. No. 5,108,597, the contents of both of which are herein incorporated by reference. It will be appreciated that other methods can also be used to create metal oxide particles. One example is an oil emulsion technique as described in Robichaud et al., Technical Note, “An Improved Oil Emulsion Synthesis Method for Large, Porous Zirconia Particles for Packed- or Fluidized-Bed Protein Chromatography,” Sep. Sci. Technol. 32, 2547-59 (1997). A second example is the formation of metal oxide particles by polymer induced colloidal aggregation as described in M. J. Annen, R. Kizhappali, P. W. Carr, and A. McCormick, “Development of Porous Zirconia Spheres by Polymerization-Induced Colloid Aggregation-Effect of Polymerization Rate,” J. Mater. Sci. 29, 6123-30 (1994). A polymer induced colloidal aggregation technique is also described in U.S. Pat. No. 5,540,834, the contents of which are herein incorporated by reference.

Metal oxide catalysts used in embodiments of the invention can be sintered by heating them in a furnace or other heating device at a relatively high temperature. In some embodiments, the metal oxide is sintered at a temperature of about 160° C. or greater. In some embodiments, the metal oxide is sintered at a temperature of about 400° C. or greater. In some embodiments, the metal oxide is sintered at a temperature of about 600° C. or greater. Sintering can be done for various amounts of time depending on the desired effect. Sintering can make metal oxide catalysts more durable. In some embodiments, the metal oxide is sintered for more than about 30 minutes. In some embodiments, the metal oxide is sintered for more than about 3 hours. However, sintering also reduces the surface area. In some embodiments, the metal oxide is sintered for less than about 1 week.

In some embodiments, the metal oxide catalyst is in the form of particles. Particles within a desired size range can be specifically selected for use as a catalyst. For example, particles can be sorted by size using techniques such as air classification, elutriation, settling fractionation, or mechanical screening. In some embodiments, the size of the particles is greater than about 0.2 μm. In some embodiments, the size range selected is from about 0.2 μm to about 10 mm. In some embodiments, the size range selected is from about 0.2 μm to about 5 mm. In some embodiments, the size range selected is from about 0.2 μm to about 1 mm. In some embodiments, the size range selected is from about 1 μm to about 100 μm. In some embodiments, the size range selected is from about 5 μm to about 15 μm. In some embodiments, the average size selected is about 10 μm. In some embodiments, the average size selected is about 5 μm.

In some embodiments, metal oxide particles used with embodiments of the invention are porous. By way of example, in some embodiments the metal oxide particles can have an average pore size of about 30 angstroms to about 2000 angstroms. However, in other embodiments, metal oxide particles used are non-porous.

The physical properties of a porous metal oxide can be quantitatively described in various ways such as by surface area, pore volume, porosity, and pore diameter. In some embodiments, metal oxide catalysts of the invention can have a surface area of between about 1 and about 400 m²/gram. In some embodiments, metal oxide catalysts of the invention can have a surface area of between about 1 and about 200 m²/gram. Pore volume refers to the proportion of the total volume taken up by pores in a material per weight amount of the material. In some embodiments, metal oxide catalysts of the invention can have a pore volume of between about 0.01 mL/g and about 2 mL/g. Porosity refers to the proportion within a total volume that is taken up by pores. As such, if the total volume of a particle is 1 cm³ and it has a porosity of 0.5, then the volume taken up by pores within the total volume is 0.5 cm³. In some embodiments, metal oxide catalysts of the invention can have a porosity of between about 0 and about 0.8. In some embodiments, metal oxide catalysts of the invention can have a porosity of between about 0.3 and 0.6.

Metal oxide particles used with embodiments of the invention can have various shapes. By way of example, in some embodiments the metal oxide can be in the form of spherules. In other embodiments, the metal oxide can be a monolith. In some embodiments, the metal oxide can have an irregular shape.

The Lewis acid sites on metal oxides of the invention can interact with Lewis basic compounds. Thus, in some embodiments, Lewis basic compounds can be bonded to the surface of metal oxides. However, in other embodiments, the metal oxides used with embodiments herein are unmodified and have no Lewis basic compounds bonded thereto. A Lewis base is an electron pair donor. Lewis basic compounds of the invention can include anions formed from the dissociation of acids such as hydrobromic acid, hydrochloric acid, hydroiodic acid, nitric acid, sulfuric acid, perchloric acid, boric acid, chloric acid, phosphoric acid, pyrophosphoric acid, chromic acid, permanganic acid, phytic acid and ethylenediamine tetramethyl phosphonic acid (EDTPA), and the like. Lewis basic compounds of the invention can also include hydroxide ion as formed from the dissociation of bases such as sodium hydroxide, potassium hydroxide, lithium hydroxide and the like.

The anion of an acid can be bonded to a metal oxide of the invention by refluxing the metal oxide in an acid solution. By way of example, metal oxide particles can be refluxed in a solution of sulfuric acid. Alternatively, the anion formed from dissociation of a base, such as the hydroxide ion formed from dissociation of sodium hydroxide, can be bonded to a metal oxide by refluxing in a base solution. By way of example, metal oxide particles can be refluxed in a solution of sodium hydroxide. The base or acid modification can be achieved under exposure to the acid or base in either batch or continuous flow conditions when disposed in a reactor housing at elevated temperature and pressure to speed up the adsorption/modification process. In some embodiments, fluoride ion, such as formed by the dissociation of sodium fluoride, can be bonded to the particles.

In some embodiments, metal oxide particles can be packed into a housing, such as a column. Disposing metal oxide particles in a housing is one approach to facilitating continuous flow processes. Many different techniques may be used for packing the metal oxide particles into a housing. The specific technique used may depend on factors such as the average particle size, the type of housing used, etc. Generally speaking, particles with an average size of about 1-20 microns can be packed under pressure and particles with an average size larger than 20 microns can be packed by dry-packing/tapping methods or by low pressure slurry packing In some embodiments, the metal oxide particles of the invention can be impregnated into a membrane, such as a PTFE membrane.

However, in some embodiments, metal oxide catalysts used with embodiments of the invention are not in particulate form. For example, a layer of a metal oxide can be disposed on a substrate in order to form a catalyst used with embodiments of the invention. The substrate can be a surface that is configured to contact the feedstocks during processing. In one approach, a metal oxide catalyst can be disposed as a layer over a surface of a reactor that contacts the feedstocks. Alternatively, the metal oxide catalyst can be embedded as a particulate in the surface of an element that is configured to contact the feedstocks during processing.

It is believed that the synthesis of products from lipid and water using a metal oxide catalyst is temperature dependent. If the temperature is not high enough, the synthesis reaction will not proceed optimally. If the temperature is too high, the desired product may not be created. As such, in some embodiments, the reaction is carried out at about 150° Celsius or hotter. In some embodiments, the reaction is carried out at about 200° Celsius or higher. In some embodiments, the reaction is carried out at about 300° Celsius or higher. In some embodiments, the reaction is carried out at about 150° Celsius and about 400° Celsius. In some embodiments, the the reaction is carried out at about 180° Celsius and about 220° Celsius. In some embodiments, the temperature is greater than the critical temperature for water.

In an embodiment, the pressure during the reaction is greater than the vapor pressures of any of the components of the reaction mixture. In an embodiment, the pressure is greater than about 100 psi. In an embodiment, the pressure is greater than about 500 psi. In an embodiment, the pressure is greater than about 800 psi. In an embodiment, the pressure is greater than about 1000 psi. In an embodiment, the pressure is greater than about 1500 psi. In an embodiment, the pressure is greater than about 2000 psi. In an embodiment, the pressure is greater than about 3000 psi. In an embodiment, the pressure is greater than about 3000 psi. In an embodiment, the pressure is greater than about 4000 psi. In some embodiments, the pressure is between about 1500 psi and about 5000 psi. In some embodiments, the pressure during the reaction is greater than the critical pressure of water.

In an embodiment, the contact time is between about 0.1 seconds and 2 hours. In an embodiment, the contact time is between about 1 second and 20 minutes. In an embodiment, the contact time is between about 2 seconds and 1 minute.

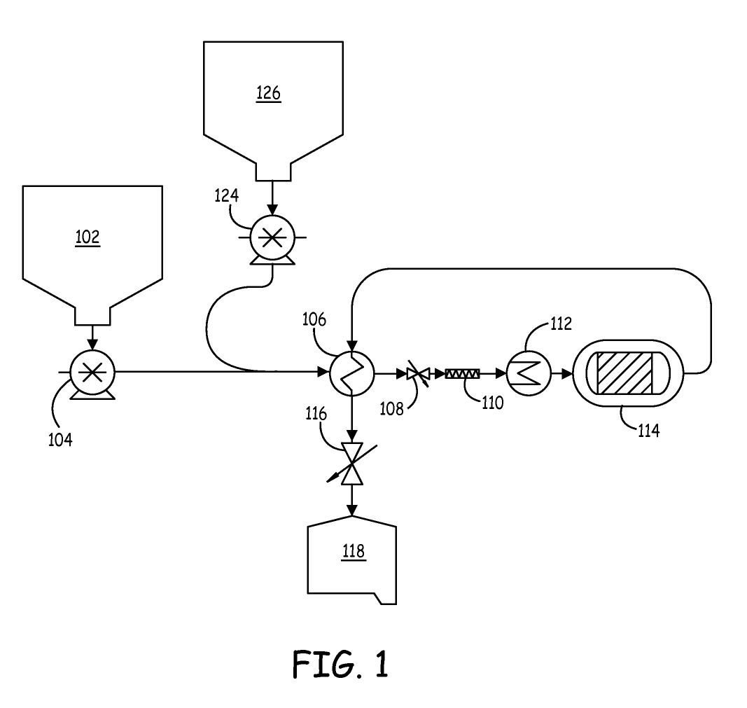

Referring now to FIG. 1, a schematic view of a reactor is presented in accordance with an embodiment of the invention. In this embodiment, a lipid feedstock is held in a lipid feedstock tank 102. Various examples of lipid feedstocks are described in greater detail below. However, it will be appreciated that the scope of lipid feedstocks contemplated for use herein is quite broad and therefore the listing is being provided only by way of non-limiting example. Water is held in a water tank 126. In some embodiments, it may simply be pure water. In other embodiments, various additives may be included with the water. In some embodiments, the water has a pH of approximately 7. In other embodiments, the water has a pH of between about 6 and 8. In some embodiments, one or both of the tanks can be heated. The lipid feedstock tank may be continuously sparged with an inert gas such as nitrogen to remove dissolved oxygen from the feedstock. Similarly, the water tank may be continuously sparged with an inert gas such as nitrogen to remove dissolved oxygen.

The feedstocks then pass from the lipid feedstock tank 102 and water tank 126 through pumps 104 and 124, respectively, before being combined and passing through a heat exchanger 106 where the feedstocks absorb heat from downstream products. The mixture then passes through a shutoff valve 108 and, optionally, a filter 110. The feedstock mixture then passes through a preheater 112 and through a reactor 114 where the feedstock mixture is converted into a product mixture. The reactor can include a metal oxide catalyst, such as in the various forms described herein. In some embodiments, the metal oxide catalyst is in the form of a particulate and it is packed within the reactor.

The reaction product mixture can pass through the heat exchanger 106 in order to transfer heat from the effluent reaction product stream to the feedstock streams. The liquid reaction product mixture can also pass through a backpressure regulator 116 before passing on to a liquid reaction product storage tank 118. Various other processes can be performed on the product mixture. By way of example, a lipid phase can be separated from a phase that includes a product mixture. In some embodiments, various products can be separated from one another using distillation techniques.

### Lipid Feed Stocks

Lipid feed stocks used in embodiments of the invention can be derived from many different sources. In some embodiments, lipid feed stocks used in embodiments of the invention can include biological lipid feed stocks. Biological lipid feed stocks can include lipids (fats or oils) produced by any type of microorganism, plant or animal. In an embodiment, the biological lipid feed stocks used include triglycerides. Many different biological lipid feed stocks derived from plants can be used. By way of example, plant-based lipid feed stocks can include rapeseed oil, soybean oil (including degummed soybean oil), canola oil, cottonseed oil, grape seed oil, mustard seed oil, corn oil, linseed oil, safflower oil, sunflower oil, poppy-seed oil, pecan oil, walnut oil, oat oil, peanut oil, rice bran oil, camellia oil, castor oil, and olive oil, palm oil, coconut oil, rice oil, algae oil, seaweed oil, Chinese Tallow tree oil. Other plant-based biological lipid feed stocks can be obtained from argan, avocado, babassu palm, balanites, borneo tallow nut, brazil nut, calendula, camelina, caryocar, cashew nut, chinese vegetable tallow, cocoa, coffee, cohune palm, coriander, cucurbitaceae, euphorbia, hemp, illipe, jatropha, jojoba, kenaf, kusum, macadamia nuts, mango seed, noog abyssinia, nutmeg, opium poppy, perilla, pili nut, pumpkin seed, rice bran, sacha inche, seje, sesame, shea nut, teased, allanblackia, almond, chaulmoogra, cuphea, jatropa curgas, karanja seed, neem, papaya, tonka bean, tung, and ucuuba, cajuput, clausena anisata, davana, galbanum natural oleoresin, german chamomile, hexastylis, high-geraniol monarda, juniapa-hinojo sabalero, lupine, melissa officinalis, milfoil, ninde, patchouli, tarragon, and wormwood.

Many different lipid feed stocks derived from animals can also be used. By way of example, animal-based biological lipid feed stocks can include choice white grease, lard (pork fat), tallow (beef fat), fish oil, and poultry fat.

Many different lipid feed stocks derived from microorganisms (Eukaryotes, Eubacteria and Archaea) can also be used. By way of example, microbe-based lipid feed stocks can include the L-glycerol lipids of Archaea and algae and diatom oils.

In some embodiments, lipid feed stocks derived from both plant and animal sources can be used such as yellow grease, white grease, and brown grease. By way of example, yellow, white or brown grease can include frying oils from deep fryers and can thus include fats of both plant and animal origin. Lipid feed stocks can specifically include used cooking oil. Brown grease (also known as trap grease) can include fats extracted from sewage systems and can thus include fats of both plant and animal origin. In some embodiments, lipid feed stocks used in embodiments of the invention can include non-biological lipid feed stocks. Lipid feed stocks of the invention can include black oil.

In some embodiments, lipid feed stocks can be derived from microorganisms such as bacteria, protozoa, algae, and fungi. Lipid feed stocks of the invention can also include soap stock and acidulated soap stock.

Lipid feed stocks used with embodiments of the invention can specifically include low value feed stocks. Low value feed stocks, such as various types of animals fats and waste oils, generally have a relatively high concentration of free fatty acids. One method of assessing the concentration of free fatty acids is to determine the acid number (or acid value) of the feed stock. The acid number is the mass of potassium hydroxide (KOH) in milligrams that is required to neutralize one gram of the chemical substance being assessed. The precise acid number as measured can vary because of the heterogeneity of the lipid feed stock. However, as an example, a high value feed stock such as virgin soybean oil can have an acid number of about 0.35 whereas a lower value feed stock such as swine tallow can have an acid number of about 5. Yellow grease, a low value feed stock, can have an acid number of about 15 while acidulated soap stock, also a low value feed stock, can have an acid number of about 88.

Systems and methods of the invention can advantageously use low value feed stocks in order to produce biodiesel fuel while achieving high percent conversion rates. In some embodiments, the lipid feed stock used has an acid number of about 3 (mg KOH/g oil) or greater. In some embodiments, the lipid feed stock used has an acid number of about 5 (mg KOH/g oil) or greater. In some embodiments, the lipid feed stock used has an acid number of about 10 (mg KOH/g oil) or greater. In some embodiments, the lipid feed stock used has an acid number of about 50 (mg KOH/g oil) or greater.

The present invention may be better understood with reference to the following examples. These examples are intended to be representative of specific embodiments of the invention, and are not intended as limiting the scope of the invention.

### EXAMPLES

### Example 1

### Formation of a Reactor

Titania catalyst was dry-packed into two of 25 cm×10.0 mm i.d. stainless steel reactor tubes. Each tube contained 27.1 g of titania (Batch no. 66-126).

### Example 2

### Production of Glycerin, Lactic Acid, and Propylene Glycol

Two 25 cm×10.0 mm i.d. reactors (from Example 1) were utilized. Two high pressure Waters 590 HPLC pumps obtained from Waters Corporation (Milford, Mass.) were used to pump water and heated oil feedstock (continuously sparged with nitrogen) into the reactor system. The oil feedstock (e.g. food grade soybean oil) was first filtered by passing the liquid through a stainless steel blank reactor fitted with two 2 micron and then into a heat exchanger where the heat from the hot effluent from the reactor was exchanged with the two incoming reactant streams.

After the heat exchanger, the two reactant streams were combined using a “T” and then passed through an electrically driven preheater that was capable of bringing the mixture to the desired set point temperature before it entered the thermostated fixed bed catalytic reactor. The backpressure of the system was maintained through the use of a backpressure regulator. Conditions were as described in Table 1 below.

TABLE 1

| | | | Contact | Molar | Total | Back- |
|---|---|---|---|---|---|---|
| Sample | | Temp | Time | Ratio | Pressure | pressure |
| ID | Feed Stock | (C.) | (min) | (TG:water) | (psi) | (psi) |
| ST36- | Refined | 300 | 1.5 | 1:40 | 2850 | 2250 |
| 61A | Soybean Oil | | | | | |
| ST36- | Refined | 310 | 1.5 | 1:40 | 2700 | 2250 |
| 61DE | Soybean Oil | | | | | |
| ST36- | Refined | 325 | 1.5 | 1:40 | 2700 | 2250 |
| 61EF | Soybean Oil | | | | | |
| ST36- | Refined | 290 | 2 | 1:40 | 2650 | 2250 |
| 60BC | Soybean Oil | | | | | |
| ST36- | Refined | 280 | 3 | 1:40 | 2600 | 2250 |
| 58AB | Soybean Oil | | | | | |
| ST36- | Refined | 260 | 3 | 1:40 | 2600 | 2250 |
| 62CD | Soybean Oil | | | | | |
| ST36- | Refined | 240 | 4.5 | 1:40 | 2600 | 2250 |
| 63DE | Soybean Oil | | | | | |

The product mixture sample was then collected and two layers (lipid and water/product) were allowed to separate spontaneously in a heated vessel kept at 70° C. using a heated water bath. The bottom layer was then centrifuged to remove any residual oil/FFAs. The centrifuged water/product solution was then distilled at 95° C. under vacuum (100 mbar) to obtain the products for each condition tested.

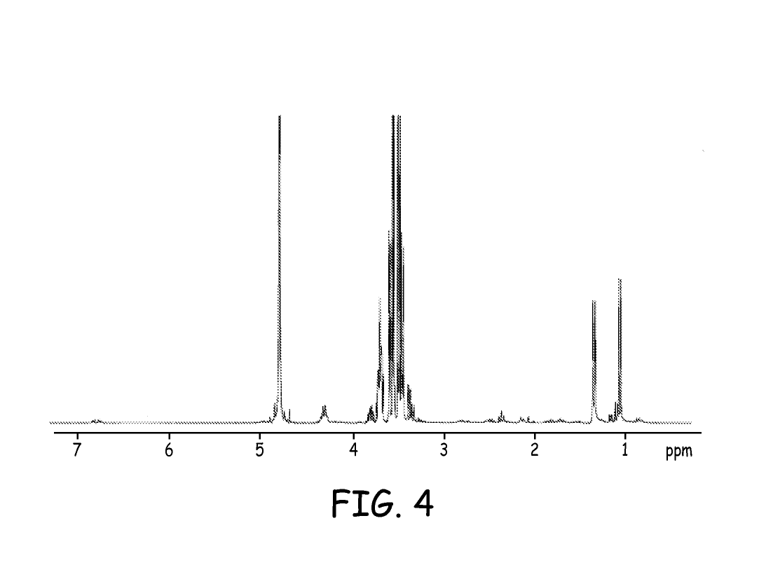

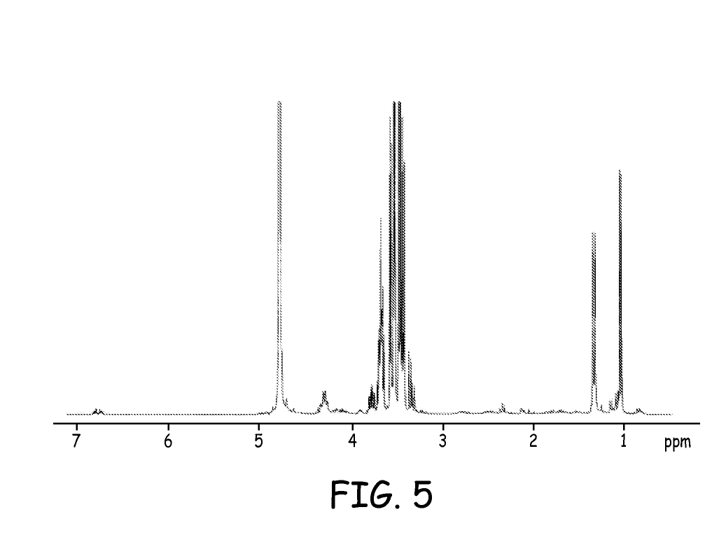

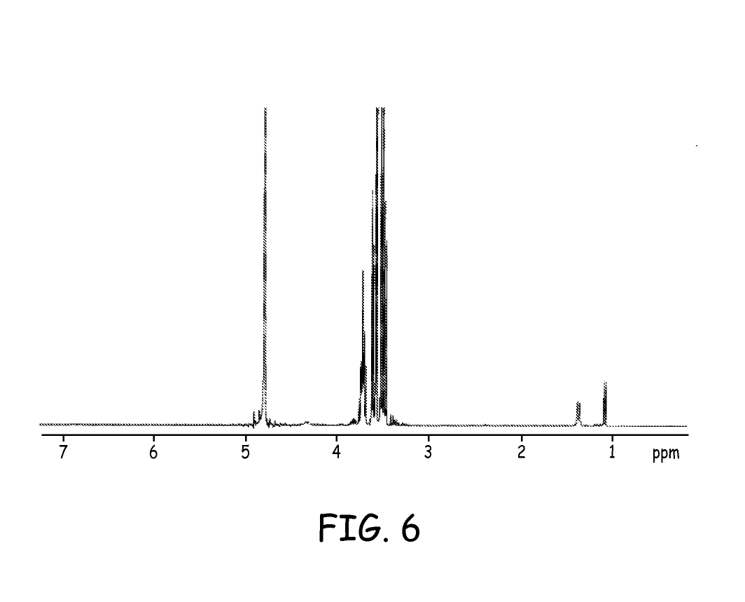

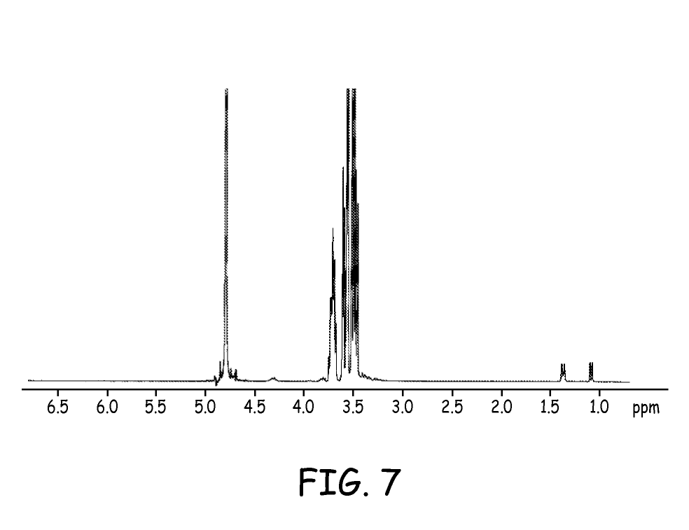

The percent yield was calculated by dividing the mass of the product collected (such as glycerin) divided by the theoretical mass contained in the lipid source (e.g. theoretically, 100 g of triglycerides contains 10 g of glycerin). The ASTM 6584 method was used to determine the glycerin content in the target product samples by GC-FID. The results for the different conditions explored are shown in Table 2 below. The acid number of the target compound (glycerin-rich) layer and the lipid layer, the purity and yield of glycerin, yield of lactic acid, and yield of propylene glycol were determined by chromatographic and spectroscopic methods. 1H-NMR was also conducted on the samples as shown in FIGS. 4-7.

ASTM 6584 method was used to derivatize the glycerin and the other compounds produced in the study in order to volatilize them for GC-MS analysis. The identity of each peak was determined using an MS spectral library. The GC-MS spectra of the samples was used to determine the composition of the product layer in each experiment.

Table 2 shows that the highest purity glycerin produced in this work was 97.8% before further treatment. The experimental conditions that were used to produce this sample were: 240° C. reactor temperature and 4.5 min contact time between the reactants and the catalyst.

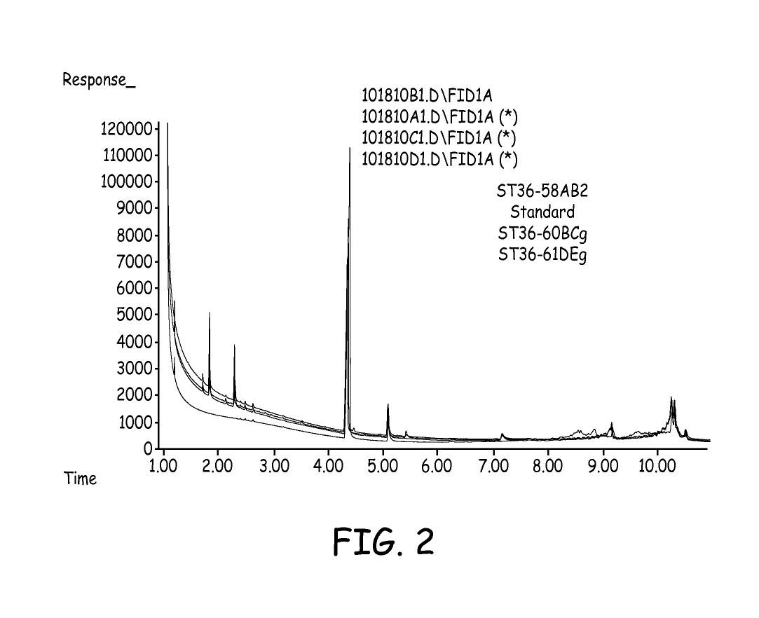

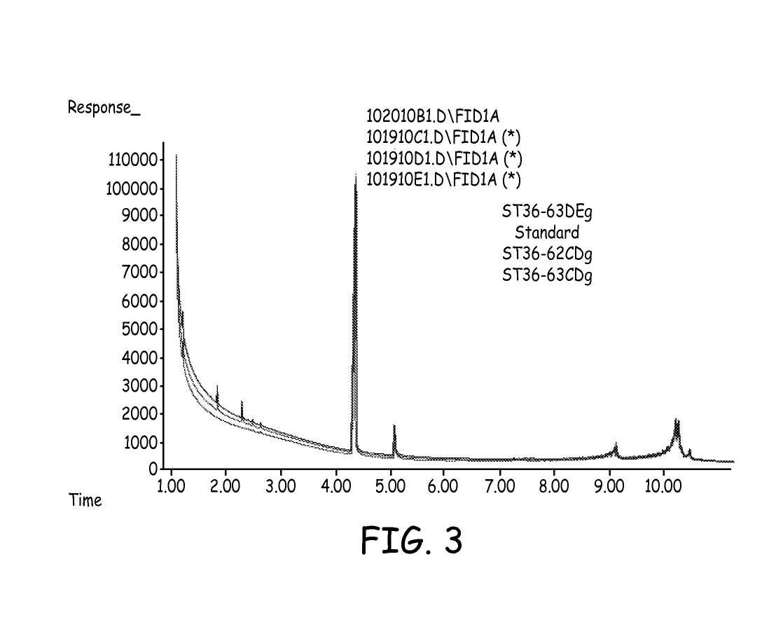

Also, as shown in Table 2, (wherein AN=acid number) there were co-products such as lactic acid and propylene glycol produced and, in general, the amount of co-products that were produced was found to increase with higher reactor temperatures. The GC chromatograms for the glycerin in samples compared to a pure glycerin standard are shown in FIGS. 2-3. Also as shown in FIG. 3, the amounts of co-products produced were significantly decreased under lower reactor temperatures and longer catalyst contact times.

TABLE 2

| | | | | | | Pro- | |
|---|---|---|---|---|---|---|---|
| | | AN of | Glyc- | Glyc- | Lactic | pylene | |
| | AN of | Glyc- | erin | erin | Acid | Glycol | |
| Sample | Oil | erin- | Yield | Purity | Yield | Yield | |
| ID | Layer | Water | % | % | % | % | Others* |
| ST36- | 100 | 3.2 | 35.3 | 83.3 | 3.7 | 4.3 | 56.7 |
| 61A | | | | | | | |
| ST36- | 110 | 4.4 | 16.9 | 53.1 | 10.4 | 7 | 65.7 |
| 61DE | | | | | | | |
| ST36- | 147 | 7.1 | 14.8 | 61.1 | 10.2 | 1.1 | 73.9 |
| 61EF | | | | | | | |
| ST36- | 147 | 7.3 | 30.5 | 59.6 | 10.5 | 9.4 | 49.5 |
| 60BC | | | | | | | |
| ST36- | 152 | 8.5 | 44.9 | 69.1 | 11.1 | 8.2 | 35.8 |
| 58AB | | | | | | | |
| ST36- | 100.4 | 1.87 | 39.3 | 84.6 | 2.7 | 2.9 | 55.1 |
| 62CD | | | | | | | |
| ST36- | 99.8 | 1.23 | 53.6 | 97.8 | 1 | 1.1 | 44.3 |
| 63DE | | | | | | | |

### Example 2

### Separation of Products in Product Mixture

Glycerol produced from the hydrolysis of a triglyceride source based on condition of ST36-63DE was concentrated by the removal of water through a rotary using a temperature of 70° C. at a reduced pressure of 40 mmHg. In order to remove the co-products, propylene glycol and lactic acid, from the glycerin, simple vacuum distillation was employed (˜5 mmHg).

As-made crude glycerol and co-products (15 g) were placed in a 100 mL round bottom flask. A 3-way adapter was attached and the top joint was secured with a rubber stopper. A water cooled condenser was attached and then a vacuum adapter. The collection flask was immersed in an ice bath. The liquid distillate was collected until a significant change in viscosity of the distillate was observed. This change in viscosity is indicative of glycerin being distilled. At this point the heat source was removed and the system cooled to room temperature. The original crude glycerin and distillate were analyzed by ¹H-NMR.

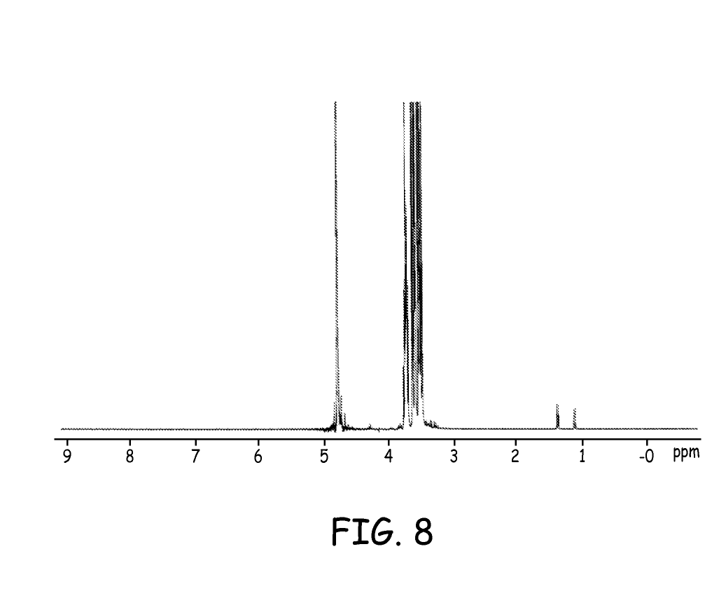

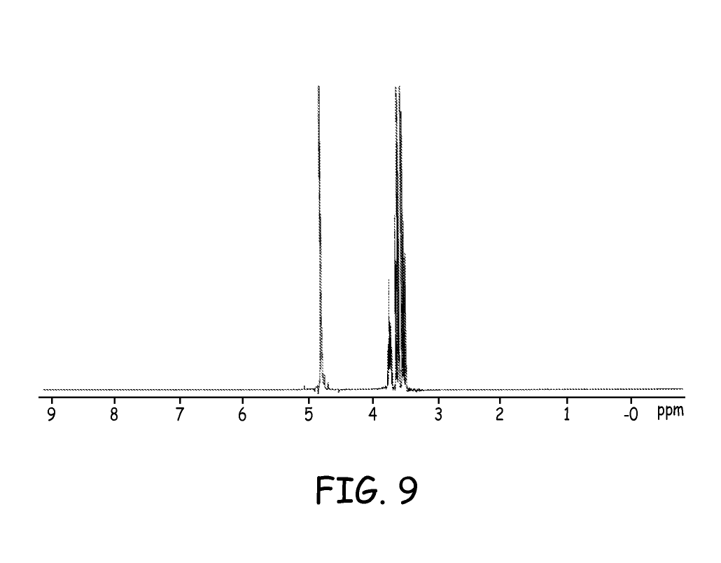

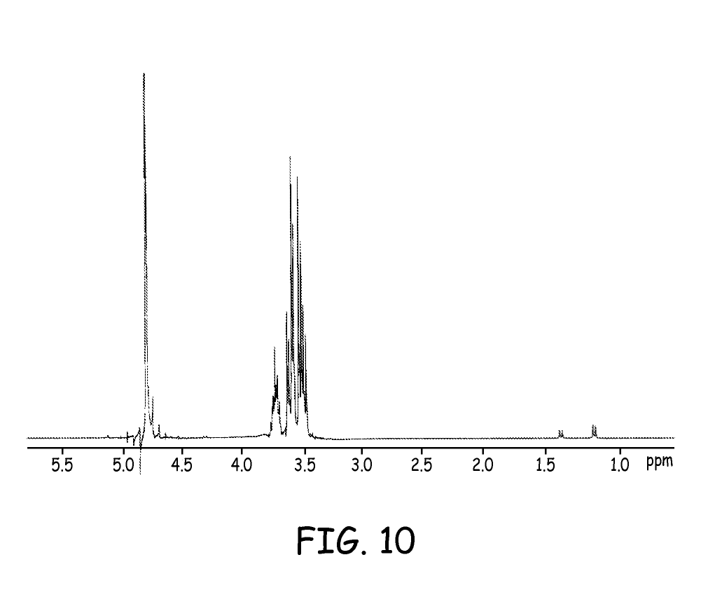

The crude glycerin spectrum is shown in FIG. 8 (resonances were assigned to δ 3.73, 3.59, 3.50 ppm), propylene glycol (δ 3.84, 3.49, 3.37, 1.08 ppm) and lactic acid (δ 4.33, 1.38 ppm). The spectrum of the remaining pot in FIG. 9 clearly shows that the lactic acid and propylene glycol were removed leaving only pure glycerin behind. The ¹H-NMR spectrum of the distillate shown in FIG. 10 which shows resonances only for glycerin. It was therefore concluded that distillation could be used in the fractionation of glycerin and the co-products produced using the process described herein.

### Further Embodiments

In an embodiment, a method of processing a lipid feedstock is included herein. The method can include combining the lipid feedstock with water to form a reaction mixture and contacting the reaction mixture with a catalyst, the catalyst comprising a metal oxide.

In an embodiment, a method of producing a polyol is included herein. The method can include combining a lipid feedstock with water to form a reaction mixture and contacting the reaction mixture with a catalyst, the catalyst comprising a metal oxide.

In an embodiment, a method of producing glycerin is included herein. The method can include combining a lipid feedstock with water to form a reaction mixture and contacting the reaction mixture with a catalyst, the catalyst comprising a metal oxide.

In an embodiment, a method of producing a diol is included herein. The method can include combining a lipid feedstock with water to form a reaction mixture and contacting the reaction mixture with a catalyst, the catalyst comprising a metal oxide.

In an embodiment, a method of producing propylene glycol is included herein. The method can include combining a lipid feedstock with water to form a reaction mixture and contacting the reaction mixture with a catalyst, the catalyst comprising a metal oxide.

In an embodiment, a method of producing carboxylic acid is included herein. The method can include combining a lipid feedstock with water to form a reaction mixture and contacting the reaction mixture with a catalyst, the catalyst comprising a metal oxide.

In an embodiment, a method of producing alpha hydroxy acid is included herein. The method can include combining a lipid feedstock with water to form a reaction mixture and contacting the reaction mixture with a catalyst, the catalyst comprising a metal oxide.

In an embodiment, a method of producing lactic acid is included herein. The method can include combining a lipid feedstock with water to form a reaction mixture and contacting the reaction mixture with a catalyst, the catalyst comprising a metal oxide.

In an embodiment, for the various methods herein, the catalyst can include a metal oxide selected from the group consisting of alumina, titania, zirconia, and hafnia. In an embodiment, the catalyst can include an unmodified metal oxide selected from the group consisting of alumina, titania, zirconia, and hafnia. In an embodiment, the catalyst can include a metal oxide with Lewis acid and Lewis base sites. In an embodiment, the step of contacting the reaction mixture with a catalyst is performed at a temperature of between 100 degrees Celsius and 400 degrees Celsius. In an embodiment, the step of contacting the reaction mixture with a catalyst is performed at a temperature of between 200 degrees Celsius and 400 degrees Celsius. In an embodiment, the step of contacting the reaction mixture with a catalyst is performed at a temperature of greater than or equal to 100 degrees Celsius. In an embodiment, the step of contacting the reaction mixture with a catalyst is performed at a temperature of greater than or equal to 200 degrees Celsius. In an embodiment, the step of contacting the reaction mixture with a catalyst is performed at a temperature of greater than or equal to 220 degrees Celsius. In an embodiment, the step of contacting the reaction mixture with a catalyst is performed at a temperature of greater than or equal to 240 degrees Celsius. In an embodiment, the step of contacting the reaction mixture with a catalyst is performed at a temperature of greater than or equal to 260 degrees Celsius. In an embodiment, the step of contacting the reaction mixture with a catalyst is performed at a temperature of greater than or equal to 280 degrees Celsius. In an embodiment, the step of contacting the reaction mixture with a catalyst is performed at a temperature of greater than or equal to 300 degrees Celsius. In an embodiment, the step of contacting the reaction mixture with a catalyst is performed at a pressure of greater than 100 psi. In an embodiment, the step of contacting the reaction mixture with a catalyst is performed at a pressure of greater than 500 psi. In an embodiment, the step of contacting the reaction mixture with a catalyst is performed at a pressure of greater than 1000 psi. In an embodiment, the step of contacting the reaction mixture with a catalyst is performed at a pressure of greater than 1500 psi. In an embodiment, the step of contacting the reaction mixture with a catalyst is performed at a pressure of greater than 2000 psi. In an embodiment, the step of contacting the reaction mixture with a catalyst is performed at a pressure of between about 1500 and 5000 psi. In an embodiment, the step of contacting the reaction mixture with a catalyst is performed at supercritical temperature and pressure conditions for water. In an embodiment, the pH of the water is between about 6.0 and 8.0. In an embodiment, the step of contacting the reaction mixture with a catalyst is performed with a contact time of greater than 2 seconds and less than 12 hours. In an embodiment, the step of contacting the reaction mixture with a catalyst is performed with a contact time of greater than about 10 seconds and less than 60 minutes. In an embodiment, the step of contacting the reaction mixture with a catalyst is performed with a contact time of greater than 30 seconds and less than 10 minutes. In an embodiment, the step of contacting the reaction mixture with a catalyst is performed with a contact time of greater than 1 minute and less than 5 minutes. In an embodiment, the catalyst comprising particles with an average particle size of about 0.2 microns to about 10 millimeters. In an embodiment, the step of contacting the reaction mixture with a catalyst is performed with a contact time of between 1 minute and 5 minutes and at a temperature of between about 270 degrees Celsius and about 330 degrees Celsius. In an embodiment, the catalyst having a porosity of between about 0.3 and 0.6. In an embodiment, the catalyst having a pore volume of between about 0 and 0.6 mls/gram. In an embodiment, the catalyst comprising a particulate metal oxide having a surface area of between about 1 and 200 m²/gram. In an embodiment, the lipid feed stock including a component selected from the group consisting of acidulated soapstock, tall oil, rapeseed oil, soybean oil, canola oil, cottonseed oil, grape seed oil, mustard seed oil, corn oil, linseed oil, sunflower oil, poppy-seed oil, walnut oil, peanut oil, rice bran oil, camellia oil, castor oil, and olive oil, palm kernel oil, coconut oil, rice oil, algae oil, seaweed oil, Chinese Tallow tree oil, yellow grease, choice white grease, lard, tallow, brown grease, fish oil and poultry fat. In an embodiment, the lipid feed stock comprising an acid number of greater than about 0.3. In an embodiment, contacting the reaction mixture with a catalyst results in the formation of a product mixture, further comprising separating components of the product mixture. In an embodiment, the catalyst comprising a mixture of different metal oxides.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

The claims are:

1. A method of producing a polyol comprising:
combining a lipid feedstock with water to form a reaction mixture;
contacting the reaction mixture with a catalyst, the catalyst comprising a metal oxide.

2. The method of claim 1, the polyol comprising glycerin.

3. The method of claim 1, wherein the step of contacting the reaction mixture with a catalyst is performed at a temperature of between 200 degrees Celsius and 400 degrees Celsius.

4. The method of claim 1, wherein the step of contacting the reaction mixture with a catalyst is performed at a pressure of between about 1500 and 5000 psi.

5. The method of claim 1, wherein the step of contacting the reaction mixture with a catalyst is performed at supercritical temperature and pressure conditions for water.

6. The method of claim 1, wherein the pH of the water is between about 6.0 and 8.0.

7. The method of claim 1, the catalyst comprising particles with an average particle size of about 0.2 microns to about 10 millimeters.

8. The method of claim 1, wherein the step of contacting the reaction mixture with a catalyst is performed with a contact time of between 1 minute and 5 minutes and at a temperature of between about 270 degrees Celsius and about 330 degrees Celsius.

9. The method of claim 1, the lipid feed stock including a component selected from the group consisting of acidulated soapstock, tall oil, rapeseed oil, soybean oil, canola oil, cottonseed oil, grape seed oil, mustard seed oil, corn oil, linseed oil, sunflower oil, poppy-seed oil, walnut oil, peanut oil, rice bran oil, camellia oil, castor oil, and olive oil, palm kernel oil, coconut oil, rice oil, algae oil, seaweed oil, Chinese Tallow tree oil, yellow grease, choice white grease, lard, tallow, brown grease, fish oil and poultry fat.

10. The method of claim 1, the lipid feed stock comprising an acid number of greater than about 0.3.

11. The method of claim 1, wherein contacting the reaction mixture with a catalyst results in the formation of a product mixture, further comprising separating components of the product mixture.

12. The method of claim 1, the catalyst comprising a metal oxide selected from the group consisting of alumina, titania, zirconia, and hafnia.

13. The method of claim 1, the catalyst comprising an unmodified metal oxide selected from the group consisting of alumina, titania, zirconia, and hafnia.

14. A method of producing a diol comprising:
combining a lipid feedstock with water to form a reaction mixture;
contacting the reaction mixture with a catalyst, the catalyst comprising a metal oxide.

15. The method of claim 14, the diol comprising propylene glycol.

16. The method of claim 14, the catalyst comprising a metal oxide selected from the group consisting of alumina, titania, zirconia, and hafnia.

17. The method of claim 14, the catalyst comprising an unmodified metal oxide selected from the group consisting of alumina, titania, zirconia, and hafnia.

18. A method of producing a carboxylic acid comprising:
combining a lipid feedstock with water to form a reaction mixture;
contacting the reaction mixture with a catalyst, the catalyst comprising a metal oxide.

19. The method of claim 18, the carboxylic acid comprising an alpha hydroxy acid.

20. The method of claim 18, the carboxylic acid comprising lactic acid.
